(19) United States
(12) Patent Application Publication
(10) Pub. No.: US 2015/0031963 A1
(43) Pub. Date: Jan. 29, 2015

(54) SYSTEM, METHOD AND DEVICE FOR MEASURING A GAS IN THE STOMACH OF A MAMMAL

(71) Applicants: Andre-Denis Wright, Campbell (AU); Keith Ellis, Campbell (AU); Joy Dempsey, Campbell (AU); Leslie Overs, Campbell (AU); Philip Valencia, Campbell (AU); David Paull, Campbell (AU); Chris McSweeney, Campbell (AU)

(72) Inventors: Andre-Denis Wright, Campbell (AU); Keith Ellis, Campbell (AU); Joy Dempsey, Campbell (AU); Leslie Overs, Campbell (AU); Philip Valencia, Campbell (AU); David Paull, Campbell (AU); Chris McSweeney, Campbell (AU)

(73) Assignees: Meat & Livestock Australia Limited, North Sydney (AU); Australian Wool Innovation Limited, Sydney (AU); Commonwealth Scientific and Industrial Research Organisation (CSIRO), Campbell (AU)

(21) Appl. No.: 14/129,688

(22) PCT Filed: Jun. 29, 2012
(86) PCT No.: PCT/AU2012/000784

§ 371 Date: May 27, 2014

(30) Foreign Application Priority Data
Jul. 1, 2011 (AU) 2011902611
Jul. 1, 2011 (AU) 20111902610
Sep. 8, 2011 (AU) 2011903645

(51) Int. Cl. A61B 5/00; A61B 5/01; G01N 1/22; A61B 5/03; A61B 5/07; G01N 33/497

(52) U.S. Cl. CPC A61B 5/4238; A61B 5/076; G01N 33/497; A61B 5/073; G01N 1/2226; A61B 5/036; A61B 5/01; G01N 2033/4977; G01N 2033/4975; G01N 2001/2232
USPC 600/301; 600/302

(57) ABSTRACT

A gas measurement device for measuring at least one gas in the stomach of a mammal, the device comprises a housing for being located in the mammal's stomach and providing at least one gas sensor for detecting the gas. The housing is impermeable to liquid within the stomach. The device may also comprise a controller disposed within the housing and which is electrically coupled to the at least one gas sensor. The controller is arranged to periodically process an output from the gas sensor(s) to provide data indicative of the amount of the gas within the stomach. The controller can include a wireless transmitter for transmitting the data to a remotely located receiving device disposed exterior to the mammal.

### FIELD OF THE INVENTION

This present invention relates to a system, method and device for measuring a gas in the stomach of a mammal and more particularly, but not exclusively, to measurement of one or more greenhouse gas emissions from ruminants.

### BACKGROUND OF THE INVENTION

Over the past decade there has been a great deal of attention paid to the issue of global warming and the detrimental effect it has on the planet. Greenhouse gases, such as methane and carbon dioxide, are known to be a major cause of global warming and significant efforts are being made to mitigate such greenhouse gas emissions, particularly anthropogenic emissions. Cattle and sheep emit quantities of methane and carbon dioxide gas as a digestive by-product. In Australia, for example, methane emissions from ruminant livestock account for over 70% of agricultural methane emissions and at least 11% of the net emissions of carbon dioxide equivalents.

The livestock industry has invested large amounts of time and funds into developing mitigation strategies for reducing ruminant greenhouse gas emissions, particularly methane emissions. However, in order to develop, monitor and validate such mitigation strategies it is necessary to be able to readily measure enteric gas emissions from large numbers of individual animals. It is desirable to measure gas emissions in an autonomous fashion which does not significantly disturb or impede the animals in their natural grazing environment.

The most widely adopted technique for such free-ranging methane measurements in individual animals involves estimating the rate at which livestock exhale methane using a sulphur-hexafluoride (SF₆) tracer gas. More specifically, the technique involves placing a permeation device that releases SF₆ in the rumen of the animal. The animal is then fitted with a sampling system, typically around their neck, which is arranged to collect exhaled air from the mouth and nostrils over an extended period of time. The air sample is analysed for methane and SF₆ and these concentrations along with the known release rate allow calculation of the methane emission rate.

However, such tracer based measurement techniques have been found to generate relatively inaccurate readings. For example, some tests have shown large variability in recordings between and within animals when measured on consecutive days. One of the causes for such variability can be attributed to dust or water entering and blocking the capillary tubing within the sampling system, or through leaks in the PVC yokes utilised to retain the sampling system about the animal's neck. Another cause can be attributed to the non-uniform rate of release of the tracer gas which can greatly influence the results.

### SUMMARY OF THE INVENTION

In accordance with a first aspect of the invention there is provided a gas measurement device for measuring at least one gas in the stomach of a mammal, the device comprising:

a housing for being located in the stomach and providing at least one gas sensor for detecting a gas, the housing being impermeable to liquid within the stomach; and

a controller disposed within the housing and electrically coupled to the at least one gas sensor, the controller being arranged to periodically process an output from the at least one gas sensor to provide data indicative of the amount of the gas within the stomach, and the controller including a wireless transmitter for transmitting the data to a remotely located receiving device disposed externally of the mammal.

Typically, the mammal is a ruminant and the device is for being swallowed by the ruminant, the housing of the device including a retaining means preventing the device from being expelled from the stomach of the ruminant.

The retaining means, can for example, comprise one or more wings retained in an initial position to facilitate swallowing of the device by the ruminant and for being released to project outwardly from the housing in the ruminant's stomach.

Typically, each gas sensor is disposed within the housing and the housing comprises at least one gas permeable portion for entry of the gas into the housing from the stomach for detection by the gas sensor(s).

Typically, the gas permeable portion is a gas permeable membrane which is impermeable to the liquid within the stomach. In an embodiment, the membrane is a bi-directional membrane to accommodate changes in gas concentration in the rumen and thereby responsive to a state of flux in such an environment.

Typically, the housing has one or more openings for passage of the at least one gas into the housing and the at least one membrane covers the openings. The membrane may, for example, be formed of siloxane, polydimethyl siloxane or some other suitable gas permeable material.

Typically, the device includes two gas sensors, with each of the gas sensors being adapted to detect a different gas to one another. A gas measurement device embodied by the invention may also comprise at least one of a temperature and/or pressure sensor wherein the outputs of the temperature and/or pressure sensor are evaluated when determining the amount of the gas within the mammal's stomach.

The housing may take the form of a tubular bolus formed of a liquid impermeable material such as polypropylene.

A sacrificial material can also be located within the housing for preventing acidic gas corrosion of the at least one sensor.

Moreover, in at least some embodiments the controller further comprises a memory arranged to store a plurality of gas sensor readings wherein the readings are transmitted to the remote receiving device periodically in batches.

Typically, a power source is located within the housing for powering at least one of the controller and sensor(s).

In accordance with another aspect of the invention. there is provided a method for measuring at least one gas in the stomach of a mammal, the method comprising:

detecting the gas utilising a gas measurement device disposed within the stomach of the mammal, the device comprising a housing providing at least one gas sensor for detecting the gas and the housing being impermeable to liquid in the stomach; and

periodically processing an output from the at least one gas sensor to provide data indicative of the amount of the gas within the stomach; and

wirelessly transmitting the data to a remotely located receiving device disposed externally to the mammal.

In some forms, a method embodied by the invention may further comprise providing a ratio from the outputs of the at least one sensor to determine a relative amount of one the gases where more than one gas is being measured.

A method in accordance with the invention may also comprise the further step of correlating the determined gas amounts with gas readings indicative of gas levels emitted from the mammal and utilising the correlated data to evaluate a gas emission level for the mammal.

Hence, in another aspect of the invention there is provided a method for predicting greenhouse gas emissions for ruminants, the method comprising:

obtaining data indicative of an amount of at least one gas within the stomach of a ruminant, the data being derived from the output of at least one gas sensor provided by a gas measurement device disposed within the ruminant's stomach;

correlating the received data with emitted gas data obtained from one or more respiration chamber readings for the ruminant; and

processing the correlated data to predict a greenhouse gas emission for the ruminant.

The one or more gases detected in accordance with the invention may be selected from the group consisting of methane and carbon dioxide amongst others.

In another aspect there is provided a system for measuring at least one gas in the stomach of at least one mammal, the system comprising one or more devices embodied by the invention, and a central controller located remotely of the mammal and arranged to wirelessly communicate with respective of the devices to receive the sensed data.

Typically, the system further comprises an inter-mediate wireless repeater arranged to communicate the sensed data to the central controller.

The central controller may further comprise a-processor arranged to process the received data to evaluate the emission of the gas from respective of the mammals.

In another aspect of the invention there is provided a bolus comprising:

a tubular body for being retained in the stomach of a mammal and providing one or more gas sensors for detecting a gas within the mammals stomach;

a gas permeable membrane arranged to cover an opening in the tubular body, the opening being provided for entry of the gas into the interior of the tubular body for detection by the one or more gas sensors.

In another aspect there is provided computer program code comprising at least one instruction which, when executed by a processor, implements a method embodied by the invention.

In another aspect there is provided a computer readable medium comprising the program code embodied by the invention.

In yet another aspect there is provided a data signal comprising a computer program code embodied by the invention.

### BRIEF DESCRIPTION OF THE ACCOMPANYING DRAWINGS

Embodiments of the present invention will now be described, by way of example only, with reference to the accompanying drawings, in which:

FIG. 1 is a schematic of a system for implementing an embodiment of the present invention;

FIGS. 2a and 2b show an exploded and assembled view, respectively, of the gas measurement device of FIG. 1 in accordance with an embodiment;

FIG. 3 is a block diagram of a controller implemented by the device of FIG. 2;

FIG. 4 is a process flow illustrating method steps for measuring gas utilising the system of FIG. 1; and

FIG. 5 is a table showing test data outputted by the device of FIG. 2.

### DETAILED DESCRIPTION OF EXEMPLARY EMBODIMENTS OF THE INVENTION

The following embodiments are described in the context of a system, device and method for measuring ruminant greenhouse gas emissions and specifically, methane and carbon dioxide gas emissions.

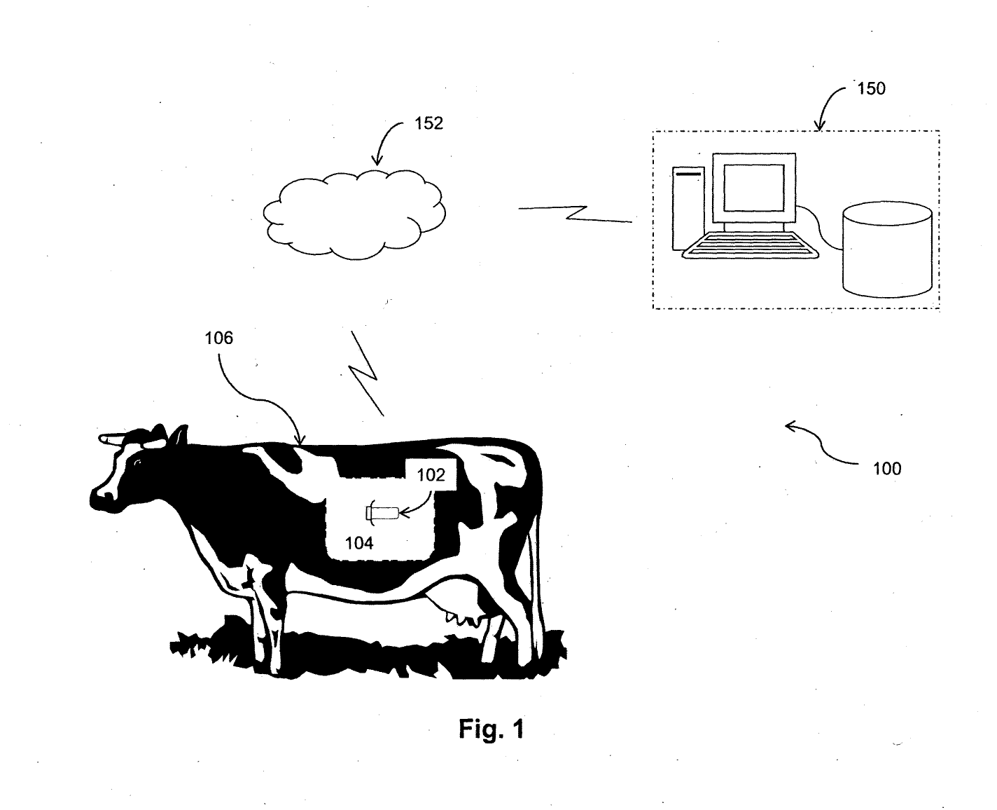

With reference to FIG. 1 there is shown a schematic illustration of a system 100 utilised to take intra-stomach greenhouse gas emission measurements, in accordance with the above-described embodiment. Utilising the proposed system 100, enteric methane and carbon dioxide gas measurements can readily be taken from large numbers of grazing ruminants (in the presently described embodiment being in the form of cattle), essentially without impeding on the animals normal grazing habits. Such a system advantageously overcomes or may ameliorate the limitations of conventional gas measurement technologies (such as respiration chamber techniques and the SF₆ tracer method described in the background) which make it difficult to reliably measure genetic and field variability and the effects of diverse farm management practices.

The system 100 includes a gas measurement device 102 for measuring both methane and carbon dioxide gas concentrations in the rumen 104 of the stomach of an animal 106. As afore-described, it will be understood that embodiments should not be seen as being limited to measuring these two gases only and could be modified to additionally or alternatively monitor other gases present within the stomach (e.g. hydrogen, oxygen, hydrogen sulphide, ammonia, etc.), depending on the desired application. Equally, depending on the desired application, the device 102 may be configured to measure only a single gas (e.g. methane).

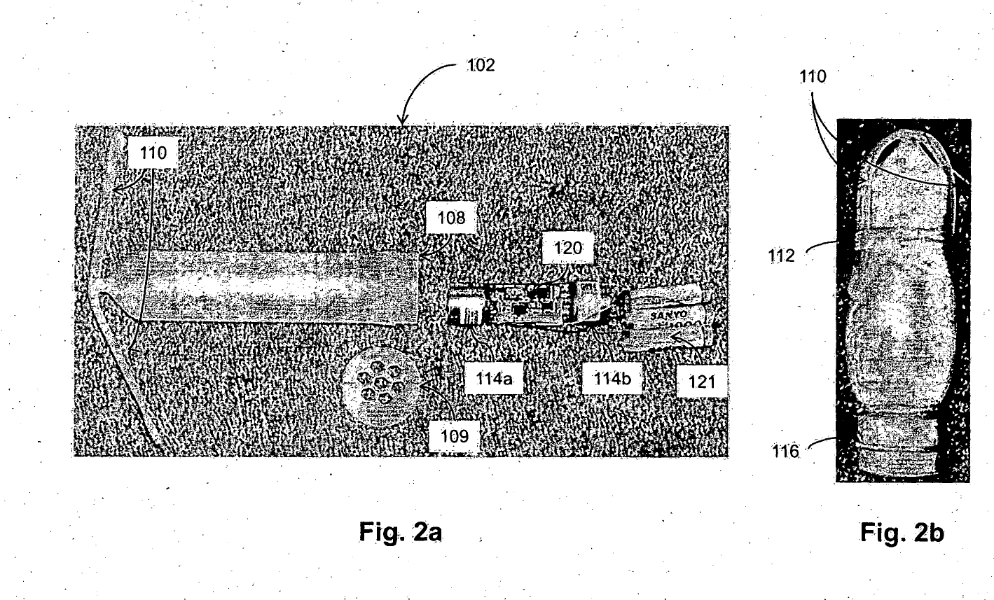

With additional reference to FIG. 2, the device 102 includes a housing in the form of a tubular bolus 108 which is designed to be retained within the rumen 104. The bolus 108 is impermeable to liquid in order to protect the electronic components therein (described in more detail in subsequent paragraphs) from water and digesta present within the rumen 104. In the presently described embodiment, the bolus 108 is formed of high density polypropylene and includes a pair of outwardly biased wings 110 which are initially held in a constrained or “closed” state by way of a dissolvable ring 112 (e.g., an elastic band), to facilitate the swallowing of the device 102 by the animal 106. This is best shown in FIG. 2b. Once the ring 112 has dissolved, the wings 110 are arranged to project outwardly (see FIG. 2a.) to prevent the device 102 from being expelled from the rumen 104. A removable end cap 109 is disposed at a distal end of the bolus 108 and is provided with a plurality of openings for reasons explained in subsequent paragraphs.

Located within the bolus 108 is a pair of gas sensors 114a, 114b for detecting the intra-ruminal methane and carbon dioxide gases respectively. Temperature and/or pressure sensors may additionally be located within the bolus 108 with their outputs utilised in the gas concentration calculations, as will be well understood by persons skilled in the art. In the presently described embodiment the sensors 114 are in the form of miniaturised non-dispersive infrared sensors, such as the TDS0035 sensor manufacture by Dynament Ltd (Derbyshire, United Kingdom; http://www.dynamet.com/) or the IR15TT-R sensor manufactured by e2v technologies (Essex, United Kingdom; http://www.e2v.com/). The sensors 114 are each arranged to measure the respective gases from 0 to 100% concentration, in increments of 0.01%. Whilst the polypropylene bolus 108 is permeable to gas, the presently illustrated embodiment includes a gas permeable portion in the form of a membrane 116 which is also impermeable to liquid within the rumen 104, and which is located across openings provided in the end cap 109 to allow for faster gas diffusion rates into the interior of the bolus (in turn allowing for more dynamic gas readings by the sensors 114a, 114b). The membrane may also or alternatively be arranged to cover holes or slits disposed along the barrel of the bolus 108 to provide greater surface area for gas diffusion. The gas permeable membrane 116 may be formed of any suitable material, although in the embodiment described herein it is made of a siloxane material and preferable polydimethyl siloxane, which has been found to suitably withstand the rigours of the rumen environment and bonds well to the polypropylene bolus 108. The membrane 116 is best shown in FIG. 2b. The gas permeable membrane can be bonded to the exterior or interior of the bolus in any suitable manner to provide a liquid impermeable barrier to entry of liquid into the bolus, such as with the use of an appropriate adhesive, or by sonic or heat welding techniques or the like. In other embodiments, the membrane 116 may be fabricated from, for instance, Kraton polymers™, low density polyethylene films or a copolymer of polypropylene and polyethylene film, polyurethanes and styrene-ethylene-butylene-styrene copolymers.

To minimise gas diffusion effects in the interior of the bolus 108, respective of the gas sensors are desirably mounted within the bolus so as to be situated as close to the gas permeable membrane as practicable. In at least some embodiments, each gas sensor may be mounted within the bolus immediately behind a different gas permeable membrane. That is, through openings may be provided in different regions of the bolus wherein the openings in each of the regions are covered by a respective gas permeable membrane, and a different one of the gas sensors is disposed within the bolus behind each of the membranes. For instance, in this embodiment, one gas sensor may be arranged at one end of the interior of the bolus and another gas sensor located at the opposite end. Also, in a particular embodiment, the gas permeable membrane may be a bi-directional membrane to accommodate for changes in gas concentration in the rumen and thereby responsive to a state of flux in such an environment.

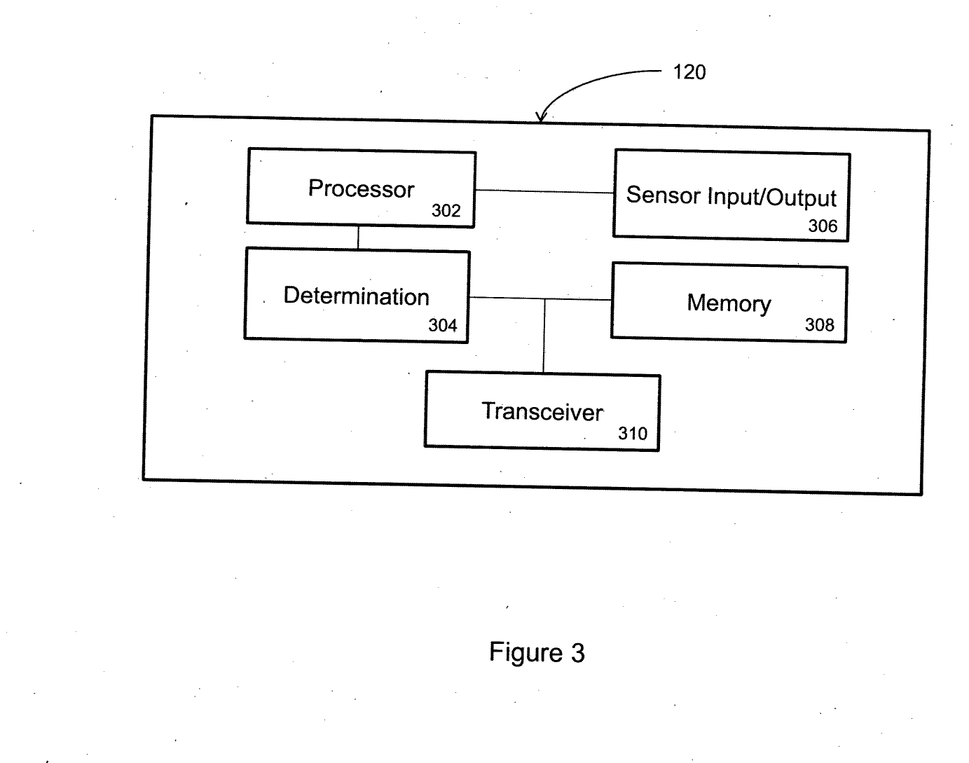

An electronic device controller 120 in the form of a Nano microcontroller manufactured by the Commonwealth Scientific and Industrial Research Organisation (CSIRO, Australia) (details of which can be found at URL http://www.ict.csiro.au/) is electrically coupled to the sensors 114, as is best shown in FIG. 2a. Power is supplied to the device controller 120 by way of three rechargeable 1.5v AAA batteries 121 locatable within the bolus 108 or any other battery type suitable for this application. With reference to the schematic of FIG. 3, the device controller 120 includes a processor 302 which is arranged to implement various modules for determining, and communicating to a central controller 150 (FIG. 1), data indicative of an amount of the respective gases within the rumen 104, based on the sensor outputs. According to the presently described embodiment, a determination module 304 is arranged to determine a percentage concentration of the two gases based on outputted voltage levels received from the sensors via input module 306. It will be appreciated that other parameters, such as molarity, may also be estimated by the determination module 304, depending on the desired application and sensor configuration. As will be understood, rather than the amount of the relevant gases being determined by the determination module 104, the amount of the gas(es) can be determined by a central controller 150 described further below that is disposed remotely from the animal(s). For the estimation of molarity or other concentration parameter of the each gas measured, an estimate of the internal volume of the rumen may be utilised.

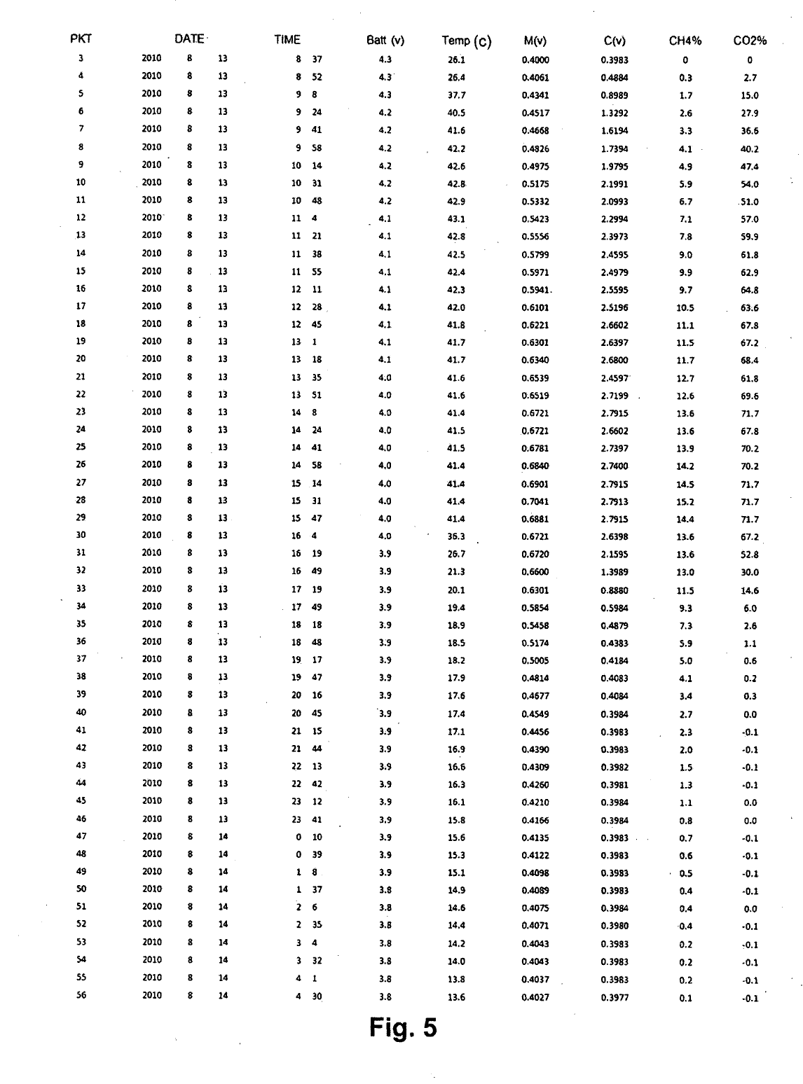

The device controller 120 also includes a flash memory 308 for logging the methane and carbon dioxide measurements in association with a date and time stamp. The device controller 120 may also log temperature, pressure and battery voltage with the gas measurements. FIG. 5 shows an example table listing raw data downloaded from a device 102 showing 56 readings taken initially at fifteen minute intervals for a rumen-fistulated sheep.

A transceiver 310 is arranged to transmit the logged data to the central controller 150 for subsequent processing and analysis, as will be described in more detail in subsequent paragraphs. In the illustrated embodiment the transceiver 310 communicates with the central controller 150 over a wireless network in the form of a radio network 152. More specifically the central controller 150 is in the form of a laptop computer enabled with a USB mounted antenna which is arranged to communicate with the transceiver 310 over the 915 MHz ISM frequency band. It will be understood that in alternative embodiments, the central controller 150 may be embodied in a server computer system arranged to communicate with the transceiver 310 over any suitable form of private or public wireless network including, for example, the GSM mobile communications network.

The transceiver 310 is arranged to transmit the logged data to the central controller 150 either in real time or in batches (e.g. when it is established that the transceiver is in wireless range of the central controller 150). The transceiver 310 is also arranged to communicate with the central controller 150 for receiving adjustment instructions. For example, the central controller 150 may send an instruction to the device controller 120 to adjust the sampling time period for the gas sensors (e.g. to adjust the sampling period from several minutes to several hours while the animal is at pasture, for preserving battery life). Likewise, the central controller 150 may send an instruction to the device controller to be on standby for an indefinite period of time until the controller reactivates the sensors in sample mode which is another method of preserving battery life. The transceiver functionality also advantageously allows the central controller 150 to interrogate a particular device 120 where multiple devices are simultaneously in operation, for example in a herd situation.

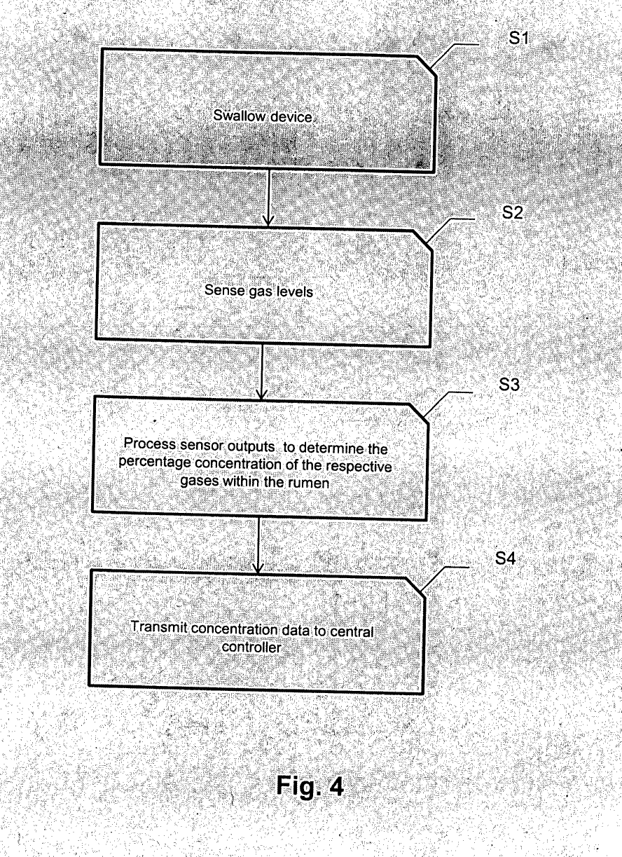

A method for measuring intra-rumen gas emissions utilising the above system 100 will now be described with reference to the flow chart 400 of FIG. 4. In a first step (S1), the device 102 is swallowed by the animal 106. After a short period of time, acids, digestive juices, enzyme(s) and/or liquids present within the rumen 104 cause the ring 112 to dissolve in turn allowing the wings 110 to project outwardly so as to modify the size and shape of the bolus thereby preventing the bolus 108 from being regurgitated during rumination from the gut and into the mouth and being expelled from the oral cavity. Upon instruction from the device controller 120 the sensors 114a, 114b are actuated for sensing methane and carbon dioxide gas levels within the rumen 104 (step S2). At step S3 the sensor outputs are processed, by the device controller 120 to determine the percentage concentrations of the respective gases within the rumen 104. At step S4, the concentration data is transmitted to the central controller 150 (either instantaneously, or in batches as previously described).

The central controller 150 is arranged to process the data received from the device 102 to provide an estimate of the greenhouse gas emissions for the animal 106. In one or more embodiments, this can involve correlating the data provided by the gas measurement device 102 with gas data output obtained from ruminant(s) of the same type in a sealed respiration chamber (which is under various experimental and grazing conditions), and utilising the correlated data to predict an emission level of the greenhouse gas or gases from the grazing field animal(s). More particularly, in the current system the infrared gas sensors 114a, 114b measure a change in voltage differential (0.4V-2.4V) as the gas concentration increases from 0-100% in a linear manner. The gas concentration for the Dynament gas sensors 114a, 114b is calculated in the following formula: gas concentration (%)=((sensor voltage reading−0.4)/2)*100. The respiration chambers estimate gas emissions from animals placed within for a set period of time (usually 24 hours) by periodically sampling air being drawn through the chamber. A variable speed electric fan draws air through a 35 mm diameter opening in the front of the chamber and then past the animal and into a similar sized manifold situated on the back wall of the chamber. A flexible plastic hose (35 mm diameter) mounted onto the rear manifold draws air into a flow meter for determination. A small diameter hose (2 mm) samples air prior to entering the flow meter and runs this sample via a multiplexer into a gas analyser for methane, carbon dioxide, hydrogen and oxygen. Each chamber can be sampled sequentially and gas concentration calculated every six minutes for just two chambers and every 15 minutes for six chambers, depending on the number of chambers in operation at any one time. Software provided by Columbus Instruments as part of their “Oxymax calorimeter System” package calculates gas emissions incorporating gas concentration and air flow rate through the system. The subsequent data are produced in a table and is also plotted on a graph for all measured gases over time as well as cumulated gas readings for the 24 hour period of animal experimentation.

The outputs from the respective gas sensors 114 can be utilised to provide an estimate of the amount of the respective individual gases measured or a ratio of the amount of one of the gases relative to the other. A ratio is particularly useful for providing an indication of the impact of changed feed, grazing, pasture or farm conditions or the like on the emission of one or more greenhouse gases by the animal(s). Likewise, by determining a ratio of one gas to another as described above, useful information can nevertheless be provided without the need to determine actual concentrations of each of the gases.

Whilst the above embodiments have been described in connection with the measurement of the greenhouse gases carbon dioxide (CO₂) and/or methane (CH₄), any other gases may be measured in the stomach of the relevant mammal. For example, non-greenhouse gases that may be measured in accordance with the invention include, for instance, ammonia (NH₃), oxygen (O₂), hydrogen (H) and hydrogen sulphide (H₂S). In one specific non-limiting example, the device 102 may implement sensors arranged to determine the concentration of ammonia in the rumen. As will be understood by persons skilled in the art, ammonia concentration in the rumen is an end product of microbial metabolism reflecting the amount of nitrogenous compounds in the rumen undergoing degradation and the nitrogen degrading activity of the rumen microbiota. Thus, the ammonia concentration determined by the device 102 can be used to interpret nitrogen input to the rumen and rate of degradation which are important for determining efficiency of nitrogen use in the rumen and potential nitrogen excretion.

In an alternative embodiment to that described above, rather than wirelessly transmitting the gas emission data to the central controller 150 the data (which are stored in memory 308) may instead be downloaded from the device 102 by way of a physical connection. For example, the device 102 may be located in a fistulated animal and once sufficient data has been obtained it is removed through a fistula in the animal and connected to a computer by way of a USB port. It will be appreciated that other techniques for physically downloading data from the device 102 are within the purview of the skilled person.

The ruminant may be any member of the order Artiodactyla, non-limiting examples of which include cattle, sheep, goats, giraffes, water buffalo, deer, camels, alpacas, llamas, elk, yak and moose. Moreover, whilst devices and methods embodied by the invention are particularly suitable for measurement of gas emissions by ruminants, it will be understood that devices and methods of the invention can equally be utilised for taking measurements of any form of gas within the stomach of other mammals. For example, embodiments may extend to measuring gas concentrations within the stomach of a member of the porcine, canine, feline, or primate family, or for instance, the stomach of a human.

In addition, although the invention has been described with reference to the present embodiments, it will be understood by those skilled in the art that alterations, changes and improvements may be made and equivalents may be substituted for the elements thereof and steps thereof without departing from the scope of the invention. Further, many modifications may be made to adapt the invention to a particular situation without departing from the central scope thereof. Such alterations, changes, modifications and improvements, though not expressly described above, are nevertheless intended and implied to be within the scope and spirit of the invention. The above described embodiments are therefore not to be taken as being limiting in any respects.

Any reference to prior art contained herein is not to be taken as an admission that the information is common general knowledge of the skilled addressee in Australia or elsewhere.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1-24. (canceled)

25. A gas measurement device for measuring a gas in the stomach of a mammal, the device comprising:
a housing for being located in the stomach, the housing being impermeable to liquid within the stomach and comprising a gas permeable portion being a gas permeable membrane for entry of the gas into the housing;
at least one gas sensor disposed within the housing for detecting the gas; and
a controller disposed within the housing and electrically coupled to the at least one gas sensor, the controller being arranged to periodically process an output from the at least one gas sensor to provide data indicative of the amount of the gas within the stomach, and the controller including a wireless transmitter for transmitting the data to a remotely located receiving device disposed externally of the mammal.

26. A device according to claim 25, wherein the mammal is a ruminant and the device is for being swallowed by the ruminant, the housing of the device including a retaining means for preventing the device from being expelled from the stomach of the ruminant.

27. A device according to claim 26, wherein the retaining means comprises one or more wings retained in an initial position to facilitate swallowing of the device by the ruminant and for being released to project outwardly from the housing in the ruminant's stomach.

28. A device according to claim 25, wherein the housing has one or more openings for passage of the gas into the housing and the gas permeable membrane covers the opening(s).

29. A device in accordance with claim 25, wherein the gas permeable membrane is a siloxane membrane.

30. A device according to claim 25, including two gas sensors, the gas sensors being adapted to detect a different type of gas present within the stomach of the mammal to one another.

31. A device according to claim 30, wherein the detectable gases comprise one or more of the following: methane, carbon dioxide, ammonia, hydrogen, hydrogen sulphide and oxygen.

32. A method for measuring a gas in the stomach of a mammal, the method comprising:
detecting the gas utilising a gas measurement device disposed within the stomach of the mammal, the device comprising a housing being impermeable to liquid within the stomach and comprising a gas permeable portion being a gas permeable membrane which permits entry of the gas into the housing for detecting by at least one gas sensor located within the housing; and
periodically processing an output from the at least one gas sensor to provide data indicative of the amount of the gas within the stomach; and
wirelessly transmitting the data to a remotely located receiving device disposed externally to the mammal.

33. A method according to claim 32, further comprising utilising two or more of the gas sensors, the gas sensors being adapted to detect a different type of gas to one another.

34. A method according to claim 33, further comprising comparing a ratio of the outputs from at least two said sensors to determine a relative amount of the detected gases within the stomach.

35. A method according to claim 32, further comprising measuring at least one of a pressure and temperature within the stomach, the pressure and/or temperature being evaluated together with the sensor output(s) for determination of the amount of the gas within the stomach.

36. A method according to claim 32, comprising the further step of correlating the determined gas amount(s) with gas readings emitted from the mammal and utilising the correlated data to evaluate a gas emission level for the mammal.

37. A method according to claim 32 wherein the gases detectable by the gas sensor(s) comprise one or more of the following: methane, carbon dioxide, ammonia, hydrogen, hydrogen sulphide and oxygen.

38. A method for predicting greenhouse gas emissions from ruminants, the method comprising:
obtaining data indicative of an amount of at least one gas within the stomach of a ruminant, the data being derived from the output of at least one gas sensor provided by a gas measurement device disposed within the ruminant's stomach;
correlating the received data with emitted gas data obtained from one or more respiration chamber readings for the ruminant; and
processing the correlated data to predict a greenhouse gas emission for the ruminant.

39. A method according to claim 38, wherein the data is indicative of a ratio of two or more gas levels within the stomach of the ruminant.

40. A method according to claim 38, wherein the at least one gas is selected from the group consisting of methane, carbon dioxide, ammonia, hydrogen, hydrogen sulphide and oxygen.

41. A system for measuring at least one gas in the stomach of at least one mammal, the system comprising one or more devices as defined in claim 25, and a central controller located remotely from the mammal and arranged to wirelessly communicate with respective of the devices to receive the data from the devices.

42. A system in accordance with claim 41, further comprising an inter-mediate wireless repeater arranged to communicate the data to the central controller.

43. A system in accordance with claim 41, wherein the central controller further comprises a processor arranged to process the received data to evaluate the emission of the gas from respective of the ruminants.

44. A bolus comprising:
a tubular body for being retained in the stomach of a mammal and comprising at least one opening;
a gas permeable membrane which is impermeable to liquid in the stomach of the mammal and being locatable over the opening(s) for entry of a gas into the interior of the tubular body while preventing ingress of the liquid;
one or more gas sensors disposed within the interior of the tubular body for detecting the amount of gas within the mammal's stomach.

44. A computer readable medium providing a program code comprising at least one instruction which, when executed by a processor, implements a method as defined in claim 32.
